(19) United States
(12) Patent Application Publication
(10) Pub. No.: US 2024/0197499 A1
(43) Pub. Date: Jun. 20, 2024

(54) MODULAR TRIAL IMPLANT SYSTEM

(71) Applicant: Aesculap AG, Tuttlingen (DE)

(72) Inventor: Thomas Saueressig, Tuttlingen (DE)

(21) Appl. No.: 18/539,973

(22) Filed: Dec. 14, 2023

(30) Foreign Application Priority Data
Dec. 15, 2022 (DE) 10 2022 133 453.8

(51) Int. Cl. A61F 2/46; A61F 2/32

(52) U.S. Cl. CPC A61F 2/4684; A61F 2/32; A61F 2002/30345

(57) ABSTRACT

A modular trial implant system includes a first trial implant part that releasably connects to a second trial implant part and/or an implant part to form a first trial joint implant part. The first trial implant part has a cone receptacle for accommodating a connecting cone of the second trial implant part or the implant part. The first trial implant part includes first latching elements and second latching elements that are axially offset in the cone receptacle for latchingly engaging behind connecting cones of second trial implant parts or implant parts. The first trial implant part has a receiving sleeve defining the cone receptacle. The receiving sleeve has a wall that surrounds the implant longitudinal axis and delimits the cone receptacle. The wall is indentation-free and/or perforation-free in a first region between the first latching elements and in a second region between the second latching elements.

### CROSS-REFERENCE TO RELATED APPLICATION(S)

This application claims priority under 35 U.S.C. § 119 to German Application No. 10 2022 133 453.8, filed on Dec. 15, 2022, the content of which is incorporated by reference herein in its entirety.

### FIELD

The present disclosure relates to a modular trial implant system comprising at least one first trial implant part, which is configured to releasably connect to at least one second trial implant part and/or an implant part to form at least one first trial joint implant part, wherein the at least one first trial implant part comprises a cone receptacle for accommodating a connecting cone of the at least one second trial implant part or the at least one implant part, wherein the cone receptacle defines an implant longitudinal axis, wherein the at least one first trial implant part comprises at least two first latching elements associated with one another and at least two second latching elements associated with one another, wherein the at least two first latching elements on the one hand and the at least two second latching elements on the other hand are arranged or formed axially offset in the cone receptacle relative to the implant longitudinal axis for engaging behind differently long connecting cones of second trial implant parts or implant parts.

### BACKGROUND

A trial implant system of the kind described at the outset is known, e.g., from EP 2 429 456 B1. The modular trial heads described in this publication, which form first trial implant parts in the sense of the modular trial implant system described at the outset, are, in particular, temporarily coupled to cones of shafts of hip joint endoprostheses during an implantation thereof in order to perform a trial repositioning. The shafts may be, e.g., trial implant parts that are inserted only temporarily during an operation into the body of a patient, or may also be implant parts that are intended to remain permanently in the body of a patient and are already inserted into a bone cavity of the patient.

A problem with the known trial heads is, in particular, that the projections, referred to as fingers, on the trial heads, when inserting them into a surgical site for coupling to a cone, and also again when removing the same, can catch on and pull along or otherwise transport soft tissues. In addition, a cleaning of such complex geometries when tissue or fluids accumulate in the interspaces between the fingers can only be achieved in a secure manner with manual pre-cleaning.

### SUMMARY

It is therefore an object of the present disclosure to improve a modular trial implant system of the kind described at the outset such that, in particular, it is more easily handleable.

This object is achieved, in accordance with the disclosure, in a modular trial implant system of the kind described at the outset in that the at least one first trial implant part comprises a receiving sleeve defining the cone receptacle, in that the receiving sleeve comprises a sleeve wall that surrounds the implant longitudinal axis and delimits the cone receptacle, and in that the sleeve wall is of indentation-free and/or perforation-free configuration at least in a first region between the at least two first latching elements and at least in a second region between the at least two second latching elements.

A modular trial implant system further developed in that way is able to be handled significantly better compared to the trial implant system known from EP 2 429 456 B1. In particular, because no indentations and/or perforations are provided in the first and second regions, when coupling to and also when removing the first trial implant part from a connecting cone of a second trial implant part inserted to remain temporarily in the body of a patient or of an implant part intended for permanent implantation, the problems described above do not occur, which arise in the case of a trial head known from EP 2 429 456 B1. It is namely not possible to jam and pull soft tissues without indentations and perforations in the first and second regions. In addition, the cleaning of the first trial implant part is also simplified, such that no manual pre-cleaning thereof is necessary. A mechanical cleaning with subsequent hot steam sterilization makes it possible, in particular, to use the first trial implant parts of the modular trial implant system multiple times. Overall, this results in an improved handling of the trial implant system for a surgeon. Second trial implant parts may also be formed, in particular, by rasp bodies of rasp tools. Such rasp bodies are, for example, releasably connectable to handles of the rasp tools provided for this purpose.

Favorably, the sleeve wall is completely of indentation-free and/or perforation-free configuration. In this way, the handling of the modular trial implant system can be improved, in particular, even further. Thus, in particular, no undesirable interaction with tissue and soft tissues of a patient can occur when inserting and removing the first trial implant part into the body of a patient for coupling to a connecting cone of a second trial implant part. In particular, the cleanability is also significantly improved, as already explained.

In order to improve the insertion of the first trial implant part into a body of a patient, it is advantageous if the cone receptacle comprises an insertion opening for inserting the connecting cone and if the insertion opening is delimited by a circular ring-shaped, uninterrupted insertion opening rim. Such an insertion opening rim, which is circumferential, offers no points of engagement for getting caught on tissue and soft tissues. Thus, a surgeon can securely couple a first trial implant part to a connecting cone of a second trial implant part and also remove it therefrom. Also, such a circumferential uninterrupted insertion opening rim is easy to clean.

It is favorable if the insertion opening rim defines a rim plane and if the rim plane extends transversely, in particular perpendicularly, to the implant longitudinal axis. An insertion opening rim configured in that way, in particular, practically cannot get caught on soft tissues and tissue of a patient. Thus, a surgeon can simply and securely couple the first trial implant part to a connecting cone of a second trial implant part or an implant part.

In accordance with a further preferred embodiment, provision may be made that the cone receptacle conically tapers in the direction toward a cone base, and that the cone base extends transversely, in particular perpendicularly, to the implant longitudinal axis. The cone base may serve, in particular, as a stop for a, for example planar, end face of the connecting cone. When the first trial implant part is moved at a sufficient speed with the cone base against an end face of the connecting cone, a clicking noise is produced that is audible to a surgeon. This noise signals to them that the trial implant parts are coupled to one another in the desired way. In particular, the cone base may be of perforation-free configuration.

It is advantageous if a perforation is formed on the at least one first trial implant part and if the perforation passes through the cone base. In this way, in particular, a venting of the cone receptacle can be achieved, so that, in particular, trapped air can escape from the cone receptacle, which would otherwise have to be compressed when placed on the connecting cone. Moreover, such a solution, i.e. without a perforation, would make an acoustic feedback practically impossible when snapping and latching the first trial implant part on the connecting cone of the second trial implant part or the implant part. Also, tissues and fluids can escape from the cone receptacle through the perforation, such that, in particular, an easy merging and snapping of the first trial implant part onto the connecting cone of the second trial implant part is made possible.

The modular trial implant system can be formed in a simple manner, in particular, if the perforation is arranged or formed coaxially to the implant longitudinal axis.

It is favorable if the cone base forms a stop face for a cone end face of the connecting cone, said stop face pointing in the direction toward the insertion opening. In particular, the stop face may be of annular configuration. An annular stop face can be achieved in a simple manner, in particular, if the cone base has a circular ring-shaped perforation. As already explained, the stop face can provide an acoustic feedback to a surgeon when the interacting first and second trial implant parts or the interacting first trial implant parts and implant parts are coupled to one another in a defined manner.

Preferably, a first distance of the at least two first latching elements from the cone base is smaller than a second distance of the at least two second latching elements from the cone base. This configuration makes it possible, in particular, to couple the first trial implant part to differently long connecting cones of second trial implant parts. A coupling can take place, in particular, by engaging behind the connecting cone, namely in a region in which a constriction adjoins the expanding connecting cone, as is typically provided in femoral shafts of hip joint endoprostheses or rasp bodies of modular rasps used for this. In this way, the number of required components of the modular trial implant system can be halved, since each first trial implant part can be coupled to two connecting cones of different lengths. Therefore, different first trial implant parts, for example in the form of trial heads, do not have to be provided for connecting cones of different lengths. As explained, the first trial implant parts can be coupled to two connecting cones of different lengths in a defined manner.

In order to enable a simple and secure coupling of the first trial implant part to a connecting cone of a second trial implant part or a second implant part, it is favorable if the at least two first latching elements extend in the circumferential direction relative to the implant longitudinal axis. This allows them to simultaneously engage behind a connecting cone.

Furthermore, it is favorable if the at least two first latching elements are arranged or formed evenly distributed over a circumference of the sleeve wall. If, for example, only two first latching elements are provided, these can be diametrically opposed to one another relative to the implant longitudinal axis. However, three or four first latching elements may also be provided, which are then arranged or formed offset over the circumference of the sleeve wall by corresponding offset angles, namely either 120° or 90°.

In a corresponding manner, it is advantageous if the at least two second latching elements extend in the circumferential direction relative to the implant longitudinal axis. They, too, can simultaneously engage behind a connecting cone on a second trial implant part or an implant part.

Preferably, the at least two second latching elements are arranged or formed distributed evenly over a circumference of the sleeve wall. For example, two, three, four, or more second latching elements may be provided. These can then be latched simultaneously to a connecting cone by engaging behind it when placed thereon.

For a simple and reliable coupling of the first trial implant part to a connecting cone of a second trial implant part or an implant part, it is favorable if the at least two first latching elements define a first latching element plane and if the first latching element plane extends transversely, in particular perpendicularly, to the implant longitudinal axis.

In a similar manner, it is advantageous if the at least two second latching elements define a second latching element plane and if the second latching element plane extends transversely, in particular perpendicularly, to the implant longitudinal axis.

The modular trial implant system can be formed in a simple manner if the at least two first latching elements are configured in the form of first latching projections pointing in the direction toward the implant longitudinal axis. These can, in particular, engage behind an edge in a transition region of the connecting cone to a neck region with a reduced cross section adjoining said transition region and latch into place there.

In the same way, it is advantageous if the at least two second latching elements are configured in the form of second latching projections pointing in the direction toward the implant longitudinal axis.

In accordance with a further preferred embodiment, provision may be made that the at least two first latching elements and the at least two second latching elements are arranged or formed offset from one another in the circumferential direction in such a way that, in the circumferential direction, each first latching element is arranged or formed between two second latching elements and each second latching element is arranged of formed between two first latching elements. In particular, it should be noted that the first and second latching elements lie in different latching element planes, since they are arranged or formed axially offset relative to one another with respect to the implant longitudinal axis. The offset arrangement of the first and second latching elements in the circumferential direction as proposed, in particular, makes it possible to minimize an undesirable, but not completely avoidable interaction of the first latching elements when coupling the first trial implant part to a longer connecting cone, which latches in a cooperating manner to the second latching elements. In particular, a latching, snapping, or clicking noise can be generated both when snapping the first trial implant part onto a shorter connecting cone and when snapping onto a longer connecting cone, which can serve as acoustic feedback to a surgeon that the coupling of the two trial implant parts with one another or of the first trial implant part and an implant part with one another has indeed taken place in the intended manner.

In order to simplify the production of the modular trial implant system, it is advantageous if two first latching elements are provided and if the two first latching elements are arranged or formed diametrically opposed to one another relative to the implant longitudinal axis. This is to be understood such that only two first latching elements are provided. Two first latching elements are basically sufficient to form an optimal latching/snapping connection with a connecting cone of a second trial implant part or an implant part.

Furthermore, it is favorable if two second latching elements are provided and if the two second latching elements are arranged or formed diametrically opposed to one another relative to the implant longitudinal axis. This configuration also enables a defined and secure latching of the first trial implant part to a connecting cone of a second trial implant part or an implant part.

In accordance with a further preferred embodiment, provision may be made that the at least two first latching elements extend over a first circumferential angle relative to the implant longitudinal axis, that the at least two second latching elements extend over a second circumferential angle relative to the implant longitudinal axis, and that the first circumferential angle is greater than the second circumferential angle. The smaller extent of the second latching elements in the circumferential direction results in an improved deformability of the sleeve wall in the region thereof. This has the advantage, in particular, that when inserting a longer connecting cone, the first latching elements cannot completely brake it, such that a snapping effect and an audible strike still occur when the second latching elements interact with a longer connecting cone. Such a snapping effect or the resulting clicking noise are brought about by a quick sudden contact of the cone end face of the connecting cone with the cone base. As soon as the latching elements are pushed over a rim of the connecting cone, the first trial implant part can be accelerated, such that it can strike the cone end face with the cone base with an increasing speed and thus produce a clicking noise.

It is favorable if the first circumferential angle has a value in a range of about 30° to about 50°, in particular in a range of about 35° to about 45°, and if the second circumferential angle has a value in a range of about 20° to about 30°, in particular in a range of about 22° to about 28°. Preferably, the first circumferential angle is about 40° per latching element and the second circumferential angle is about 25° per latching element. In particular, these values may be provided if only two respective first and second latching elements are provided. The circumferential angle in the specified ranges ensures, in particular, the desired functionality of the modular trial implant system.

It is favorable if the cone receptacle defines an inner wall surface, if formed on the inner wall surface is a first portion that is hollow-cylindrical relative to the implant longitudinal axis, and if the at least two first latching elements are formed on the first hollow-cylindrical portion. In particular, this further development makes it possible for the first latching elements to interact with an associated shorter connecting cone in an optimal manner. In particular, a contact of the connecting cone with the cone receptacle in the region of the first latching elements can be limited precisely to these. Contact with the hollow-cylindrical portion can be avoided by this design.

It is advantageous if formed on the inner wall surface is a second portion that is hollow-cylindrical relative to the implant longitudinal axis and if the at least two second latching elements are formed on the second hollow-cylindrical portion. In the case of the second latching elements, too, as in the case of the first latching elements in connection with the first hollow-cylindrical portion, the advantage is that here only an interaction with the connecting cone with the second latching elements, but not with the inner surface of the cone receptacle in the region of the second hollow-cylindrical portion is possible.

In accordance with a further preferred embodiment, provision may be made that a circumferential groove relative to the implant longitudinal axis is arranged or formed on the cone receptacle and that the groove is arranged or formed between the at least two first latching elements on the one hand and the at least two second latching elements on the other hand. The groove forms a weakening of the sleeve wall, which makes it more flexible or elastic in the region of the groove. This is advantageous for the function of the second latching elements. In particular, it can thus be ensured that the latching of the second latching elements to a longer connecting cone also leads to an accelerated movement of the first trial implant part when snapping onto the connecting cone in order to produce the clicking noise desired for a surgeon.

The modular trial implant system can be formed in a simple manner if the groove is concavely curved pointing in the direction toward the implant longitudinal axis. For example, it may be formed with a small radius having a value in a range of about 1 mm to about 2 mm.

It is advantageous if the groove defines a groove plane and if the groove plane extends transversely, in particular perpendicularly, to the implant longitudinal axis. For example, the groove plane can thus extend in parallel to the first and second latching element planes, in particular between them.

Preferably, the groove extends between the first hollow-cylindrical portion and the second hollow-cylindrical portion. It is not absolutely necessary for the groove to be directly adjacent to the two hollow-cylindrical portions.

The groove favorably directly adjoins the first hollow-cylindrical portion and/or directly adjoins the second hollow-cylindrical portion. Thus, in particular, it can enable an optimal effect of the at least two second latching elements in the manner described when directly adjoining the second hollow-cylindrical portion.

In accordance with a further preferred embodiment, provision may be made that the sleeve wall comprises a first sleeve portion and an adjoining second sleeve portion, that the cone receptacle tapers from the second sleeve portion in the direction toward the first sleeve portion, that the at least two first and second latching elements are arranged or formed exclusively on the second sleeve portion, and that the first wall thickness of the first sleeve portion is greater than a second wall thickness of the second sleeve portion In particular, the first wall thickness is at least about 50% greater than the second wall thickness. For example, it may be about twice as great as the second wall thickness. Forming the second sleeve portion with a reduced wall thickness has the advantage, in particular, that the sleeve wall can be easily deformed in the required manner in the region of the second sleeve portion when pushing the first trial implant part onto a connecting cone of a second trial implant part or an implant part. The first sleeve portion, on the other hand, is more stable and less elastic with the first wall thickness. Thus, the function of the first trial implant part for latching and connecting to a connecting cone of a second trial implant part or an implant part can be predetermined in a defined manner and thereby improved.

The at least one first trial implant part is favorably made of a plastic. In particular, a plastic can be used here which has the desired elasticity, in particular in the region of the second sleeve portion or in the region of the first and second latching elements. The plastic is favorably a plastic that is sterilizable with hot steam.

It is favorable if the plastic is or contains polyphenylene sulfone (PPSU). Polyphenylene sulfone is an amorphous material with a high glass transition temperature and low moisture absorption. It is therefore suitable for high-quality technical parts and highly stressed mass products. In other words, such a plastic is ideally suited for the formation of trial implant parts.

In order to enable X-ray control of the modular trial implant system, it is advantageous if the at least one first trial implant part contains an X-ray contrast agent.

Good visibility of the trial implant system under X-ray control can be achieved if the X-ray contrast agent is or contains barium sulfate.

In accordance with a further preferred embodiment, provision may be made that the modular trial implant system comprises at least one second trial implant part or at least one implant part and that the at least one second trial implant part or the at least one implant part comprises a connecting cone that is insertable into the cone receptacle. The coupled first and second trial implant parts can thus form, for example, a first trial joint implant part of an artificial trial joint.

Preferably, the modular trial implant system comprises at least two second trial implant parts or at least two implant parts with connecting cones of different lengths. As described, these can then be brought into engagement with and coupled to the first trial implant part with its first and second latching elements, respectively.

It is advantageous if the at least one second trial implant part is configured in the form of trial stem that is insertable into a bone cavity or in the form of a rasp body. For example, a bone cavity can be prepared with a rasp body. When the rasp body is inserted into the bone cavity, it can be latchingly coupled in the manner described above to a first trial implant part configured in the form of a trial head or comprising such a trial head in order to determine a size of the implant components to be implanted permanently.

The at least one first trial implant part favorably comprises a spherical or ball-shaped trial joint head. Such a trial joint head can, for example, interact with a trial joint socket of an artificial trial hip joint in order to determine the optimal size of the implant components to be implanted permanently.

The first trial implant part can be formed in a simple manner if the trial joint head has a rotationally symmetrical portion of a spherical surface relative to the implant longitudinal axis.

It is favorable if the perforation fluidically connects the cone receptacle and the spherical surface. As explained, the cone receptacle can thus be optimally ventilated. In addition, tissue and fluid can also securely escape from the cone receptacle through the perforation.

It is advantageous if the modular trial implant system comprises at least one artificial trial joint having a first trial joint implant part and a second trial joint implant part that cooperates with said first trial joint implant part in a jointed manner. With such a trial joint, the size and shape of the implant components to be ultimately implanted permanently can be reliably determined in a patient.

It is advantageous if the artificial trial joint is configured in the form of a trial hip joint, if the first trial joint implant part forms a prosthesis shaft with a trial joint head, and if the second trial joint implant part is configured in the form of a trial joint socket. In this way, using the modular trial implant system, suitable implant components can be reliably determined by a surgeon with regard to both their shape and their size for hip joint endoprostheses to be implanted permanently.

It is favorable if the at least one first trial implant part and/or the at least one second trial implant part or the implant part are of one-piece, in particular monolithic, configuration. This makes it possible to achieve a high stability of the trial implant parts. In addition, a risk can be minimized that small parts are able to fall off from the respective trial implant parts and get lost in an undesirable manner, for example in a surgical site.

The foregoing description thus comprises, in particular, the embodiments of modular trial implant systems defined in the following in the form of numbered paragraphs:
1. A modular trial implant system (12) comprising at least one first trial implant part (10), which is configured to releasably connect to at least one second trial implant part (14, 16) and/or an implant part to form at least one first trial joint implant part (18, 20), wherein the at least one first trial implant part (10) comprises a cone receptacle (22) for accommodating a connecting cone (24, 26) of the at least one second trial implant part (14, 16) or the at least one implant part, wherein the cone receptacle (22) defines an implant longitudinal axis (28), wherein the at least one first trial implant part (10) comprises at least two first latching elements (76) associated with one another and at least two second latching elements (78) associated with one another, wherein the at least two first latching elements (76) on the one hand and the at least two second latching elements (78) on the other hand are arranged or formed axially offset in the cone receptacle (22) relative to the implant longitudinal axis (28) for latchingly engaging behind differently long connecting cones (24, 26) of second trial implant parts (14, 16) or implant parts, wherein the at least one first trial implant part (10) comprises a receiving sleeve (36) defining the cone receptacle (22), wherein the receiving sleeve (36) comprises a sleeve wall (38) that surrounds the implant longitudinal axis (28) and delimits the cone receptacle, and wherein the sleeve wall (38) is of indentation-free and/or perforation-free configuration at least in a first region (96) between the at least two first latching elements (78) and at least in a second region (98) between the at least two second latching elements (78).
2. The modular trial implant system in accordance with paragraph number 1, wherein the sleeve wall (38) is completely of indentation-free and/or perforation-free configuration.
3. The modular trial implant system in accordance with paragraph number 1 or 2, wherein the cone receptacle (22) comprises an insertion opening (40) for inserting the connecting cone (24, 26) and wherein the insertion opening (40) is delimited by a circular ring-shaped, uninterrupted insertion opening rim (42).
4. The modular trial implant system in accordance with paragraph number 3, wherein the insertion opening rim (42) defines a rim plane (44) and wherein the rim plane (44) extends transversely, in particular perpendicularly, to the implant longitudinal axis (28).
5. The modular trial implant system in accordance with any one of the preceding paragraph numbers, wherein the cone receptacle (22) conically tapers in the direction toward a cone base (46) and wherein the cone base (46) extends transversely, in particular perpendicularly, to the implant longitudinal axis (28).
6. The modular trial implant system in accordance with paragraph number 5, wherein a perforation (48) is formed on the at least one first trial implant part (10) and wherein the perforation (48) passes through the cone base (46).
7. The modular trial implant system in accordance with paragraph number 6, wherein the perforation (48) is arranged or formed coaxially to the implant longitudinal axis (28).
8. The modular trial implant system in accordance with any one of paragraph numbers 5 to 7, wherein the cone base (46) forms a stop face (50) for a cone end face (52, 54) of the connecting cone (24, 26), said stop face pointing in the direction toward the insertion opening (40), wherein the stop face (50) is, in particular, of annular configuration.
9. The modular trial implant system in accordance with any one of paragraph numbers 5 to 8, wherein a first distance (88) of the at least two first latching elements (76) from the cone base (46) is smaller than a second distance (90) of the at least two second latching elements (78) from the cone base (46).
10. The modular trial implant system in accordance with any one of the preceding paragraph numbers, wherein the at least two first latching elements (76) extend in the circumferential direction relative to the implant longitudinal axis (28).
11. The modular trial implant system in accordance with any one of the preceding paragraph numbers, wherein the at least two first latching elements (76) are arranged or formed evenly distributed over a circumference of the sleeve wall (38).
12. The modular trial implant system in accordance with any one of the preceding paragraph numbers, wherein the at least two second latching elements (78) extend in the circumferential direction relative to the implant longitudinal axis (28).
13. The modular trial implant system in accordance with any one of the preceding paragraph numbers, wherein the at least two second latching elements (78) are arranged or formed evenly distributed over a circumference of the sleeve wall (38).
14. The modular trial implant system in accordance with any one of the preceding paragraph numbers, wherein the at least two first latching elements (76) define a first latching element plane (84) and wherein the first latching element plane (84) extends transversely, in particular perpendicularly, to the implant longitudinal axis (28).
15. The modular trial implant system in accordance with any one of the preceding paragraph numbers, wherein the at least two second latching elements (78) define a second latching element plane (86) and wherein the second latching element plane (86) extends transversely, in particular perpendicularly, to the implant longitudinal axis (28).
16. The modular trial implant system in accordance with any one of the preceding paragraph numbers, wherein the at least two first latching elements (76) are configured in the form of first latching projections (80) pointing in the direction toward the implant longitudinal axis (28).
17. The modular trial implant system in accordance with any one of the preceding paragraph numbers, wherein the at least two second latching elements (78) are configured in the form of second latching projections (82) pointing in the direction toward the implant longitudinal axis (28).
18. The modular trial implant system in accordance with any one of the preceding paragraph numbers, wherein the at least two first latching elements (76) and the at least two second latching elements (78) are arranged or formed offset from one another in the circumferential direction in such a way that, in the circumferential direction, each first latching element (76) is arranged or formed between two second latching elements (78) and each second latching element (78) is arranged of formed between two first latching elements (76).
19. The modular trial implant system in accordance with any one of the preceding paragraph numbers, wherein two first latching elements (76) are provided and wherein the two first latching elements (76) are arranged or formed diametrically opposed to one another relative to the implant longitudinal axis (28).
20. The modular trial implant system in accordance with any one of the preceding paragraph numbers, wherein two second latching elements (78) are provided and wherein the two second latching elements (78) are arranged or formed diametrically opposed to one another relative to the implant longitudinal axis (28).
21. The modular trial implant system in accordance with any one of the preceding paragraph numbers, wherein the at least two first latching elements (76) extend over a first circumferential angle (92) relative to the implant longitudinal axis (28), wherein the at least two second latching elements (78) extend over a second circumferential angle (94) relative to the implant longitudinal axis (28), and wherein the first circumferential angle (92) is greater than the second circumferential angle (94).
22. The modular trial implant system in accordance with paragraph number 21, wherein the first circumferential angle (92) has a value in a range of about 30° to about 50°, in particular in a range of about 35° to about 45°, and wherein the second circumferential angle (94) has a value in a range of about 20° to about 30°, in particular in a range of about 22° to about 28°.
23. The modular trial implant system in accordance with any one of the preceding paragraph numbers, wherein the cone receptacle (22) defines an inner wall surface (64), wherein formed on the inner wall surface (64) is a first portion (66) that is hollow-cylindrical relative to the implant longitudinal axis (28), and wherein the at least two first latching elements (76) are formed on the first hollow-cylindrical portion (66).
24. The modular trial implant system in accordance with paragraph number 23, wherein formed on the inner wall surface (64) is a second portion (68) that is hollow-cylindrical relative to the implant longitudinal axis (28) and wherein the at least two second latching elements (78) are formed on the second hollow-cylindrical portion (68).
25. The modular trial implant system in accordance with any one of the preceding paragraph numbers, wherein a circumferential groove (72) relative to the implant longitudinal axis (28) is arranged or formed on the cone receptacle (22) and wherein the groove (72) is arranged or formed between the at least two first latching elements (76) on the one hand and the at least two second latching elements (78) on the other hand.
26. The modular trial implant system in accordance with paragraph number 25, wherein the groove (72) is concavely curved pointing in the direction toward the implant longitudinal axis (28).
27. The modular trial implant system in accordance with paragraph number 25 or 26, wherein the groove (72) defines a groove plane (74) and wherein the groove plane (74) extends transversely, in particular perpendicularly, to the implant longitudinal axis (28).
28. The modular trial implant system in accordance with any one of the paragraph numbers 25 to 27, wherein the groove (72) extends between the first hollow-cylindrical portion (66) and the second hollow-cylindrical portion (68).
29. The modular trial implant system in accordance with any one of the paragraph numbers 25 to 28, wherein the groove (72) directly adjoins the first hollow-cylindrical portion (66) and/or directly adjoins the second hollow-cylindrical portion (68).
30. The modular trial implant system in accordance with any one of the preceding paragraph numbers, wherein the sleeve wall (38) comprises a first sleeve portion (56) and an adjoining second sleeve portion (58), wherein the cone receptacle (22) tapers from the second sleeve portion (58) in the direction toward the first sleeve portion (56), wherein the at least two first and second latching elements (76, 78) are arranged or formed exclusively on the second sleeve portion (58), and wherein a first wall thickness (60) of the first sleeve portion (56) is greater than a second wall thickness (62) of the second sleeve portion (58), in particular at least about 50% greater.
31. The modular trial implant system in accordance with any one of the preceding paragraph numbers, wherein the at least one first trial implant part (10) is made of a plastic.
32. The modular trial implant system in accordance with paragraph number 31, wherein the plastic is or contains polyphenylene sulfone (PPSU).
33. The modular trial implant system in accordance with any one of the preceding paragraph numbers, wherein the at least one first trial implant part (10) contains an X-ray contrast agent.
34. The modular trial implant system in accordance with paragraph number 33, wherein the X-ray contrast agent is or contains barium sulfate.
35. The modular trial implant system in accordance with any one of the preceding paragraph numbers, wherein the modular trial implant system (12) comprises at least one second trial implant part (14, 16) or at least one implant part and wherein the at least one second trial implant part (14, 16) or the at least one implant part comprises a connecting cone (24, 26) that is insertable into the cone receptacle (22).
36. The modular trial implant system in accordance with paragraph number 35, wherein the modular trial implant system (12) comprises at least two second trial implant parts (14, 16) or at least two implant parts with connecting cones (24, 26) of different lengths.
37. The modular trial implant system in accordance with paragraph number 35 or 36, wherein the at least one second trial implant part (14, 16) is configured in the form of a trial stem (100, 102) that is insertable into a bone cavity or in the form of a rasp body.
38. The modular trial implant system in accordance with any one of the preceding paragraph numbers, wherein the at least one first trial implant part (10) has a spherical or ball-shaped trial joint head (30).
39. The modular trial implant system in accordance with paragraph number 37, wherein the trial joint head (30) has a rotationally symmetrical portion of a spherical surface relative to the implant longitudinal axis (28).
40. The modular trial implant system in accordance with paragraph number 38, wherein the perforation (48) fluidically connects the cone receptacle (22) and the spherical surface (32).
41. The modular trial implant system in accordance with any one of the preceding paragraph numbers, wherein the modular trial implant system (12) comprises at least one artificial trial joint (104, 106) with a first trial joint implant part (18, 20) and a second trial joint implant part (108, 110) that cooperates with said first trial joint implant part in a jointed manner.
42. The modular trial implant system in accordance with paragraph number 41, wherein the artificial trial joint (104, 106) is configured in the form of a trial hip joint, wherein the first trial joint implant part (18, 20) forms a prosthesis shaft with a trial joint head, and wherein the second trial joint implant part (108, 110) is configured in the form of a trial joint socket (112, 114).
43. The modular trial implant system in accordance with any one of the preceding paragraph numbers, wherein the at least one first trial implant part (10) and/or the at least one second trial implant part (14, 16) or the implant part are of one-piece, in particular monolithic, configuration.

### BRIEF DESCRIPTION OF THE DRAWINGS

The subsequent description of preferred embodiments of the disclosure serves in conjunction with the drawings for further explanation. In the drawings:

FIG. 1 shows a first perspective view of a first embodiment of a first trial implant part in the form of a trial head with cone receptacle;

FIG. 2 shows a further perspective view of the first trial implant part from FIG. 1 in a partially broken depiction;

FIG. 3 shows a section view along line 3-3 in FIG. 2;

FIG. 4 shows an enlarged view of the region A from FIG. 3;

FIG. 5 shows a section view along line 5-5 in FIG. 2;

FIG. 6 shows an enlarged partial view of the region B from FIG. 5;

FIG. 7 shows a partially cut view of the trial head of FIG. 1 with a first embodiment of a second trial implant part with a short connecting cone;

FIG. 8 shows an enlarged partial view of the region C in FIG. 7;

FIG. 9 shows a partially cut view of the trial head from FIG. 1 in cooperation with a second embodiment of a second trial implant part with a longer connecting cone;

FIG. 10 shows an enlarged partial view of the region D from FIG. 9; and

FIG. 11 shows a section view along line 11-11 in FIG. 9.

### DETAILED DESCRIPTION

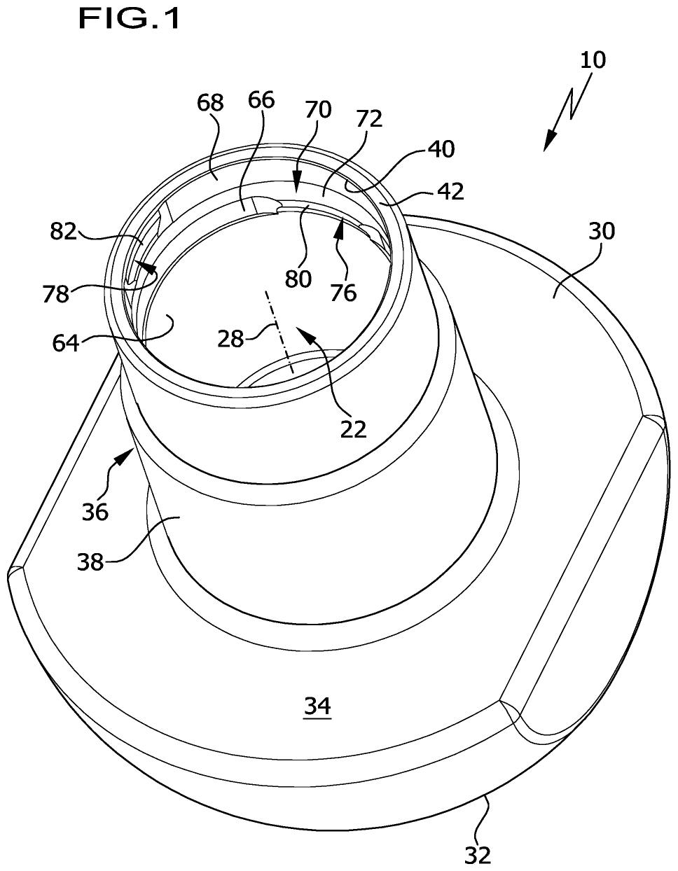

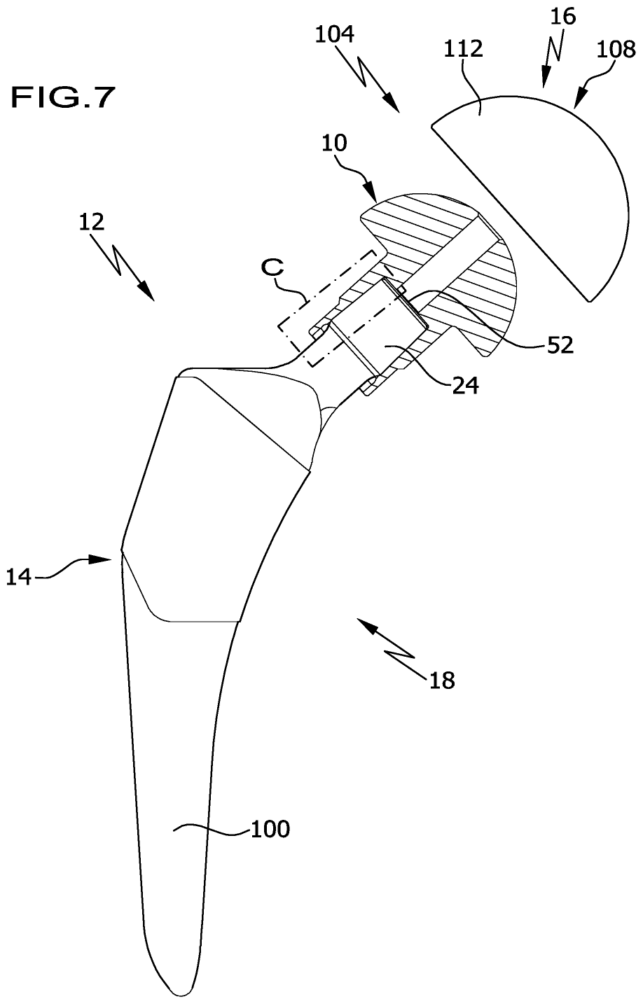

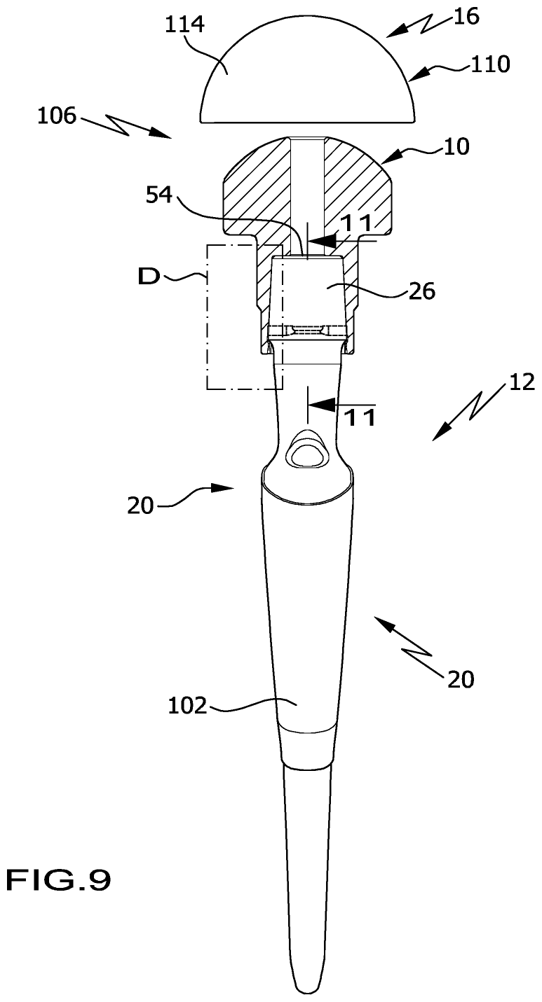

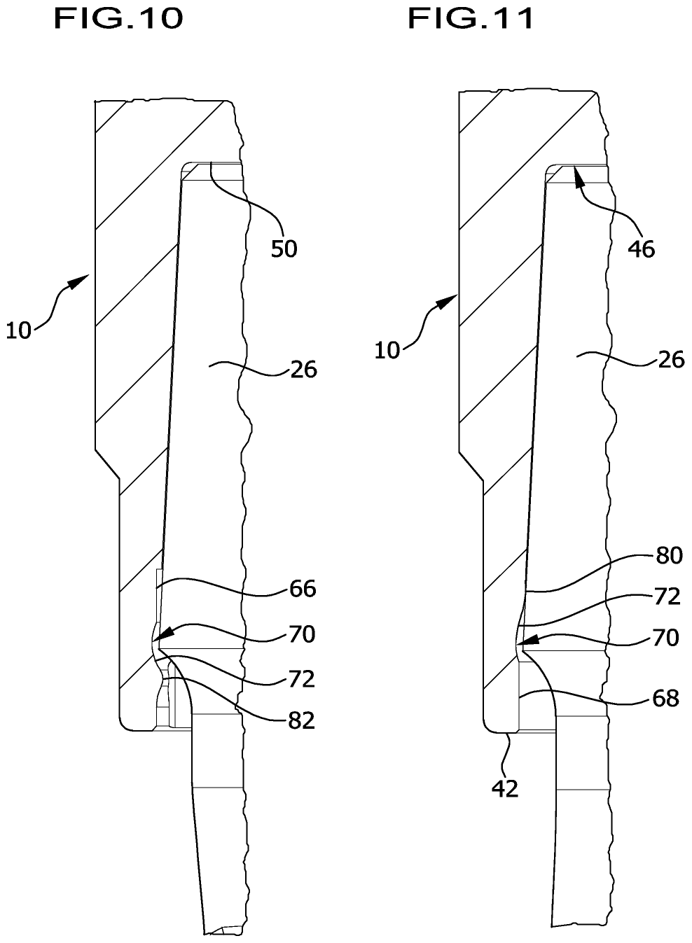

Schematically depicted in FIG. 1 is a first embodiment of a first trial implant part 10. It forms part of a modular trial implant system 12 shown schematically in FIGS. 7 and 9.

The first trial implant part 10 is configured to releasably connect to second trial implant parts 14 and 16, which are schematically depicted in FIGS. 7 and 9. The first and second trial implant parts 10 and 14 connected to one another form a first trial joint implant part 18. The first and second trial implant parts 10 and 16 connected to one another form a further first trial joint implant part 20.

The first trial implant part 10 comprises a cone receptacle 22 for accommodating a connecting cone 24 and 26 of the second trial implant part 14 and 16 respectively.

The cone receptacle 22 defines an implant longitudinal axis 28.

The first trial implant part 10 comprises a trial joint head 30. The latter is spherical or ball-shaped and overall defines a hemisphere. Laterally, the hemisphere is provided with two parallel flattened portions, which make it easier to manually handle the first trial implant part.

The trial joint head 30 defines a portion of a spherical surface 32. This portion is rotationally symmetrical relative to the implant longitudinal axis 28.

A receiving sleeve 36 extends away from a planar bottom side 34 of the trial joint head 30 coaxially to the implant longitudinal axis 28. The receiving sleeve 36 defines the cone receptacle 22. It further comprises a sleeve wall 38 that surrounds the implant longitudinal axis 28 and delimits the cone receptacle.

The cone receptacle 22 comprises an insertion opening 40 for inserting the connecting cones 24, 26. It is delimited by a circular ring-shaped, uninterrupted insertion opening rim 42. The insertion opening rim 42 defines a rim plane 44, which extends transversely, namely perpendicularly in the embodiment depicted in the Figures, to the implant longitudinal axis 28.

The cone receptacle 22 conically tapers in the direction toward a cone base 46. The cone base 46 extends transversely, namely perpendicularly in the embodiment depicted in the Figures, to the implant longitudinal axis 28.

Furthermore, a perforation 48 is formed on the first trial implant part 10, which perforation fluidically connects the cone receptacle 22 and the spherical surface 32. The perforation 48 passes through the cone base 46. It is formed coaxially to the implant longitudinal axis 28.

The cone base 46 forms a stop face 50 for cone end faces 52 and 54 of the respective connecting cones 24 and 26, said stop face pointing toward the insertion opening 40. Due to the perforation 48 passing through the cone base 46 coaxially to the implant longitudinal axis 28, the stop face 50 is of annular, namely circular ring-shaped configuration in the embodiment depicted in the Figures.

The sleeve wall 38 comprises a first sleeve portion 56 and a second sleeve portion 58. The first sleeve portion 56 extends commencing from the cone base 46 in the direction toward the second sleeve portion 58, which directly adjoins the first sleeve portion 56. In this way, the sleeve portions 56 and 58 delimit the cone receptacle 22 in such a way that the cone receptacle 22 tapers from the second sleeve portion 58 in the direction toward the first sleeve portion 56, namely up to the cone base 46.

The first sleeve portion 56 defines a first wall thickness 60. The second sleeve portion 58 defines a second wall thickness 62. The first wall thickness 60 is greater than the second wall thickness 62, namely at least about 50% greater. In the embodiment depicted in the Figures, the first wall thickness 60 is about twice as great as the second wall thickness 62.

The cone receptacle 22 defines an inner wall surface 64. The latter is conically formed commencing from the insertion opening rim 42 and tapers continuously from the insertion opening rim 42 to the cone base 46.

Deviating from the basically conical inner wall surface, a first hollow-cylindrical portion 66 and second hollow-cylindrical portion 68 are formed on said inner wall surface. The portions 66 and 68 are each of hollow-cylindrical configuration relative to the implant longitudinal axis 28. This means that the inner wall surface 64 in the regions of the first and second hollow-cylindrical portions 66 and 68 maintains its respective inner diameter and does not reduce as is the case in the remaining region of the inner wall surface 64 commencing from the insertion opening rim 42 in the direction toward the cone base 46.

The second hollow-cylindrical portion 68 directly adjoins the insertion opening rim 42.

Between the first and second hollow-cylindrical portions 66 and 68, a recess 70 in the form of a groove 72 is formed on the second sleeve portion 58. The groove 72 directly adjoins the respective hollow-cylindrical portions 66 and 68. The recess 70 and thus also the groove 72 are arranged or formed circumferentially on the cone receptacle 22 relative to the implant longitudinal axis 28. The groove 72 is also concavely curved pointing in the direction toward the implant longitudinal axis 28. Moreover, the groove 72 defines a groove plane 74, which extends transversely, namely perpendicularly in the embodiment depicted in the Figures, to the implant longitudinal axis 28.

For latchingly connecting the first trial implant part 10 to one of the connecting cones 24, 26, the first trial implant part 10 comprises two or more first latching elements 76 associated with one another and two or more second latching elements 78 associated with one another. In the embodiment of the first trial implant part 10 shown in the Figures, two respective first latching elements 76 and two respective second latching elements 78 are provided.

The first and second latching elements 76 and 78 are arranged or formed exclusively on the second sleeve portion 58.

The two first latching elements 76 are formed on the first hollow-cylindrical portion 66. By contrast, the two second latching elements 78 are formed on the second hollow-cylindrical portion 68.

The two first latching elements 76 are arranged or formed evenly distributed over a circumference of the sleeve wall 38, in particular of the first hollow-cylindrical portion 66.

The two second locking elements 78 are also arranged or formed evenly distributed over a circumference of the sleeve wall 38, thus over a circumference of the second hollow-cylindrical portion 68.

The two first latching elements 76 extend in the circumferential direction relative to the implant longitudinal axis 28. The two second latching elements 78 also extend in the circumferential direction relative to the implant longitudinal axis 28.

The two first latching elements 76 are configured in the form of first latching projections 80 pointing in the direction toward the implant longitudinal axis 28. The two second latching elements 78 are also configured in the form of second latching projections 82 pointing in the direction toward the implant longitudinal axis 28.

The even distribution of the first and second latching elements 76 and 78 over a circumference of the sleeve wall 38 leads to the two first latching elements 76 being arranged or formed diametrically opposed to one another relative to the implant longitudinal axis 28. The two second latching elements 78 are also arranged diametrically opposed to one another relative to the implant longitudinal axis 28.

Furthermore, the first latching elements 76 define a first latching element plane 84, which extends transversely, namely perpendicularly in the embodiment depicted in the Figures, to the implant longitudinal axis 28. In a similar manner, the second latching elements 78 define a second latching element plane 86, which extends transversely, namely perpendicularly in the embodiment depicted in the Figures, to the implant longitudinal axis 28. Thus, the first and second latching element planes 84, 86 extend in parallel to one another and in parallel to the cone base 46 and to the rim plane 44.

The first and second latching elements 76 and 78 are thus arranged or formed on the cone receptacle 22 axially offset from one another relative to the implant longitudinal axis 28. A first distance 88 of the first latching elements 76 from the cone base 46 is thus smaller than a second distance 90 of the second latching elements 78 from the cone base 46. Due to the different distances 88 and 90, the first and second latching elements 76 and 78 can engage behind the differently long connecting cones 24, 26 in a coupling position, as schematically depicted in FIGS. 7 and 9 for the shorter connecting cone 24 on the one hand and the longer connecting cone 26 on the other hand.

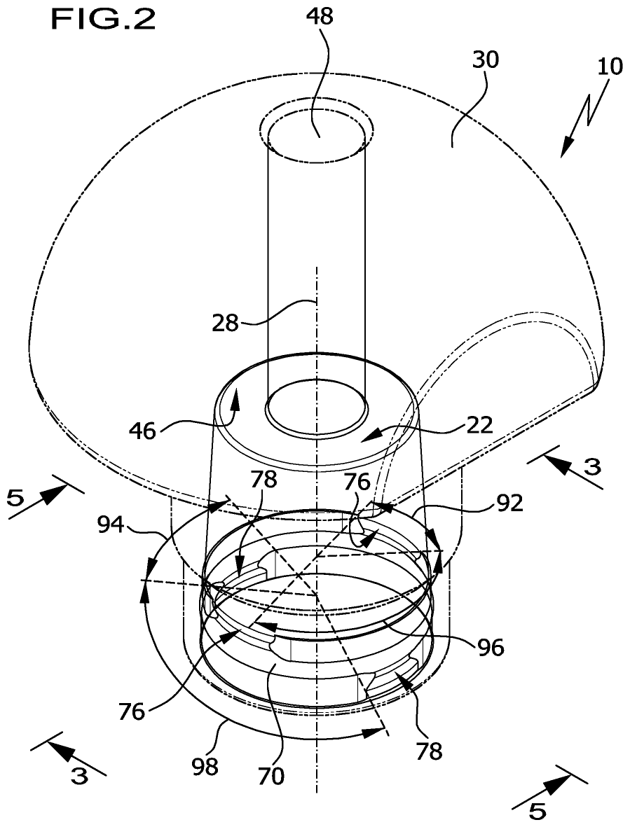

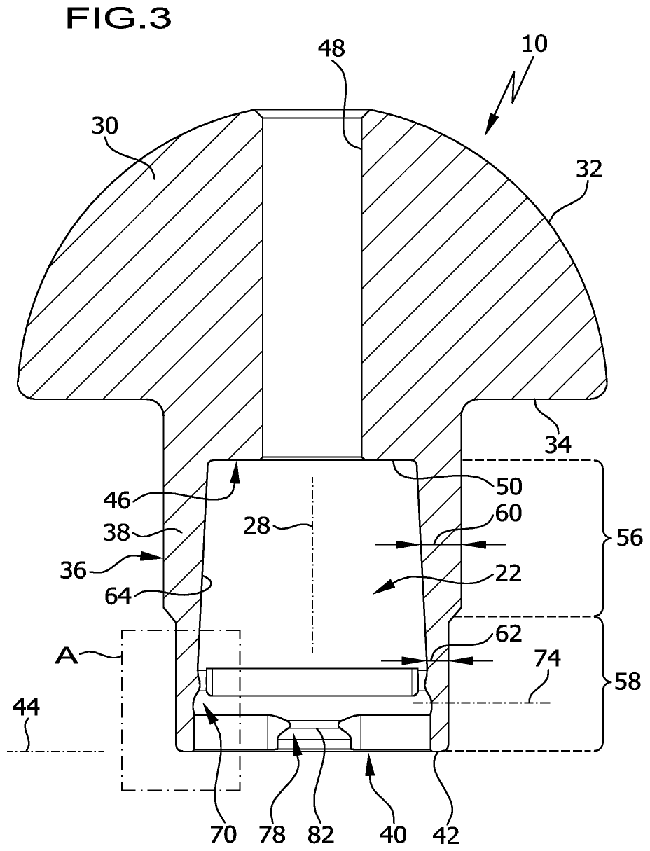

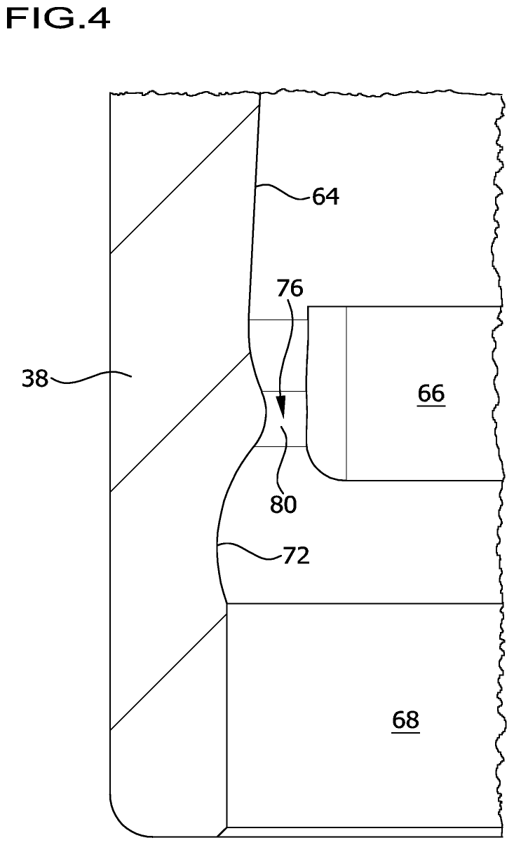

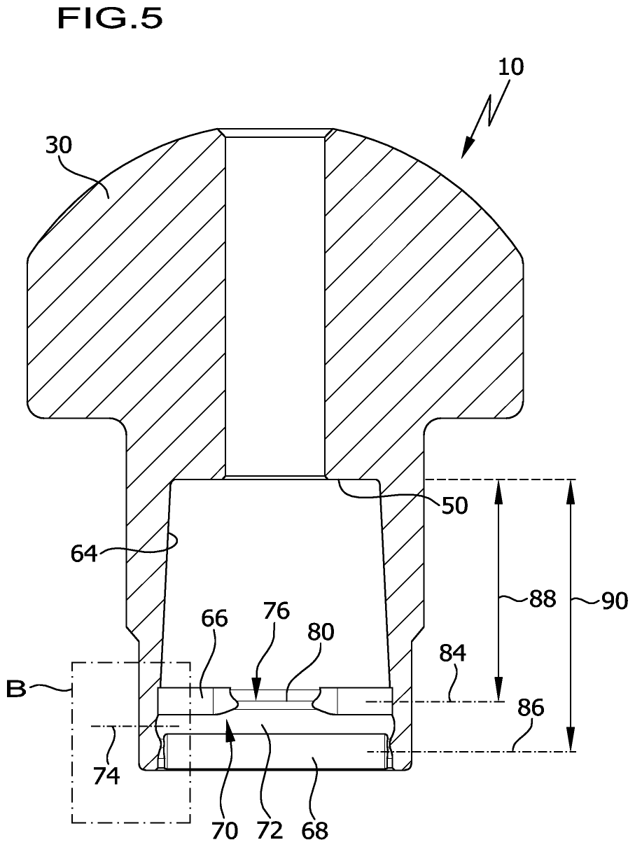

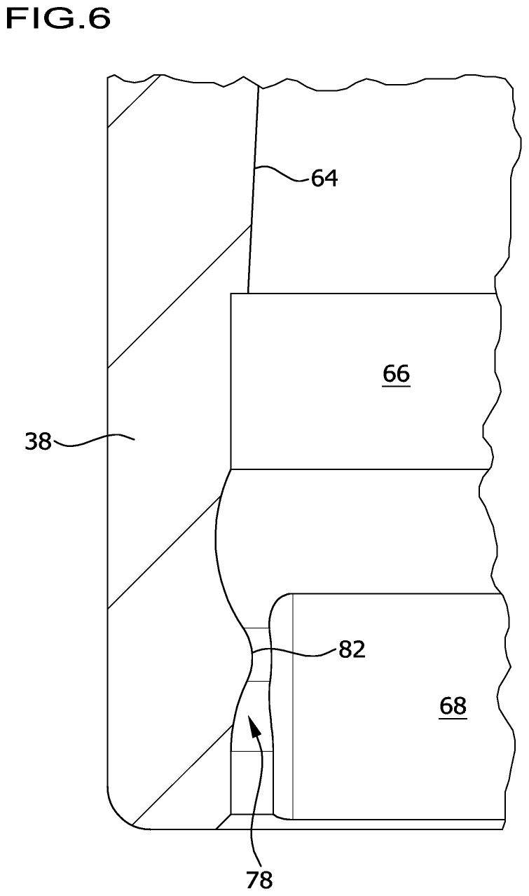

The first and second latching elements 76 and 78 are not only arranged or formed axially offset from one another relative to the implant longitudinal axis 28, but also offset from one another in the circumferential direction, namely in such a way that, in the circumferential direction, each first latching element 76 is arranged or formed between two second latching elements 78 and each second latching element 78 is arranged or formed between two first latching elements 76. This can be seen particularly well in in FIGS. 1 and 2.

The first and second latching projections 80 and 82 are formed projecting from the respective hollow-cylindrical portions 66, 68 in a bead-like manner. The first latching elements 76 each extend over a first circumferential angle 92 relative to the implant longitudinal axis 28. The second latching elements 78 each extend over a second circumferential angle 94 relative to the implant longitudinal axis 28. In the embodiment depicted in the Figures, the first circumferential angle 92 is greater than the second circumferential angle 94.

The first circumferential angle 92 has a value in a range of about 30° to about 50°. In the embodiment depicted in the Figures, the value of the first circumferential angle 92 is about 40°.

By contrast, the second circumferential angle 94 has a value in a range of about 20° to about 30°. In the embodiment depicted in the Figures, the value of the second circumferential angle 94 is about 25°.

The groove 72 extends as described between the two first latching elements 76 on the one hand and the second latching elements 78 on the other hand. The groove 72 or the recess 70 forms a weakened region of the second sleeve portion 58, thereby increasing an elasticity of the sleeve wall 38 in this region.

Thus, the first latching elements 76 together do not extend over the entire circumference of the first hollow-cylindrical portion 66. This also applies correspondingly to the second latching elements 78 with respect to the second hollow-cylindrical portion 68. In the circumferential direction, a first region 96 between the first latching elements 76 and a second region 98 between the second latching elements 78 are thus defined. In the first and second regions 96 and 98, neither indentations nor perforations are formed on the sleeve wall 38. Thus, the regions 96 and 98 are of indentation-free and perforation-free configuration.

However, the sleeve wall 38 is of indentation-free and perforation-free configuration not only in the regions 96 and 98. Rather, the sleeve wall 38 is completely of indentation-free and/or perforation-free configuration. Thus, in the first trial implant part 10, no elastic or flexible fingers are formed on which the first and second latching elements 76 and 78 are arranged or formed.

The first and second latching elements 76 and 78 are each chamfered in the circumferential direction. In the axial direction, they have an edge-free progression. Commencing from the respective hollow-cylindrical wall portions 66 and 68, the progression comprises a concave portion pointing toward the implant longitudinal axis 28, a convex portion adjoining said concave portion, and a further concave portion adjoining said convex portion. Due to this configuration, the latching elements 76 and 78 can slide on a cone outer surface of the connecting cones 24, 26, which points away from the implant longitudinal axis 28, when the connecting cones 24 and 26 are inserted into the cone receptacle 22.

The first trial implant part 10 is made of a plastic. In particular, polyphenylene sulfone (PPSU) is used as the plastic.

In order to make the first trial implant part 10 visible under X-ray control, the plastic contains an X-ray contrast agent. The latter may be, in particular, barium sulfate.

In the trial implant system 12 depicted in the Figures, the second trial implant parts 14, 16 are configured in the form of a trial stem 100 and 102 respectively, which is insertable into a bone cavity. Alternatively, second trial implant parts may also be configured in the form of rasp bodies not shown in the Figures. These may also have corresponding connecting cones, which are formed analogously to the connecting cones 24, 26 and are latchingly coupleable to the first trial implant part 10 as described.

It should be noted here that instead of the second trial implant parts 14 and 16 depicted as an example in the Figures, which are only intended to remain temporarily in the body of a patient, implant parts formed identically in shape and size that are intended to remain permanently in the body of the patient can also be used in cooperation with the first trial implant part 10 to form first trial joint implant parts of the modular trial implant system 12. These implant parts may also have corresponding connecting cones, which are formed analogously to the connecting cones 24, 26 and are latchingly coupleable to the first trial implant part 10 as described. The depiction of such implant parts was omitted in the Figures for the sake of clarity, since they do not differ in shape and size from the second trial implant parts 14 and 16 as explained.

The modular trial implant system 12 may also further comprise an artificial trial joint 104 and 106. Here, the trial joint 104 comprises the first trial joint implant part 18 and a second trial joint implant part 108 that cooperates with said first trial joint implant part in a jointed manner. The trial joint 106 comprises the first trial joint implant part 20 and a second trial joint implant part 110 that cooperates with said first trial joint implant part 20 in a jointed manner.

In FIGS. 7 and 9, the trial joints 104 and 106 are depicted as an example configured in the form of trial hip joints. Here, the first trial joint implant parts 18, 20 form a prosthesis shaft with a trial joint head and the second trial joint implant parts 108, 110 are configured in the form of a trial joint socket 112 and 114 respectively.

In the embodiment depicted in the Figures of the modular trial implant system 12, the first trial implant parts 10 and the second trial implant parts 14, 16 are each of one-piece, namely monolithic, configuration.

The functioning of the modular trial implant system 12, in particular the latching connection between the first trial implant part 10 and the second trial implant parts 14 and 16 is briefly explained below.

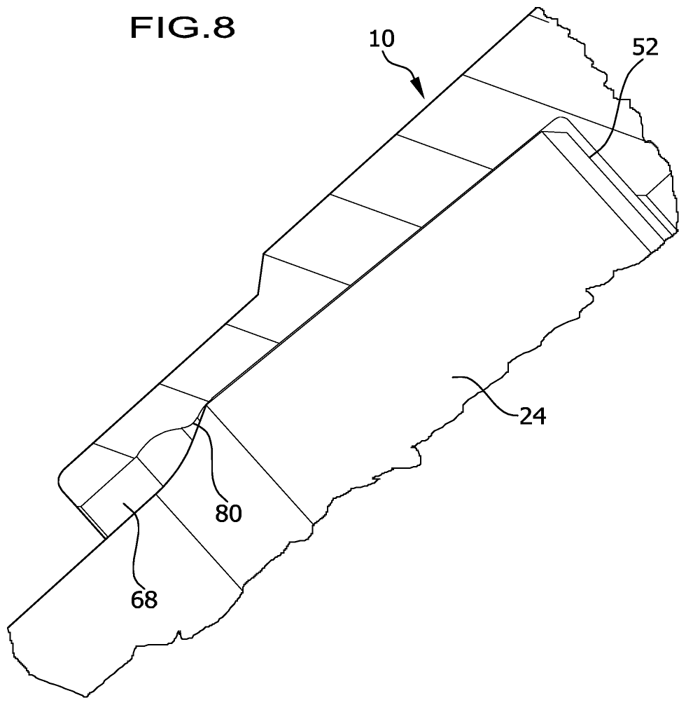

FIGS. 7 and 8 show the first trial joint implant part 18 when coupling the first and second trial implant parts 10 and 14 to one another. The connecting cone 24 is inserted into the cone receptacle 22. It has a length such that it can cooperate with the first latching elements 76. When inserting the connecting cone 24 into the cone receptacle 22, the sleeve wall 38 deforms, in particular in the region of the second sleeve portion 58 and in the region of the first hollow-cylindrical portion 66, when the first latching elements 76 slide on the outside of the connecting cone 24. When the first trial implant part 10 is pushed further onto the connecting cone 24, the first latching elements 76 can engage behind an edge on the connecting cone 24 that points away from the cone end face, the advancing force exerted on the first trial implant part 10 leading to an abrupt acceleration of the same, such that the cone base 46 is able to strike against the cone end face 52 in an unbraked manner and thus cause a clicking or snapping noise.

When the first trial implant part 10 is brought into engagement with the connecting cone 26 of the second trial implant part 16, the first latching elements 76 thus slide on the outside of the connecting cone 26. They thereby lead to a deformation of the sleeve wall 38 in the region of the second sleeve portion 58 and the first hollow-cylindrical portion 66. As soon as the second latching elements 78 are able to engage behind the longer connecting cone 26, this in turn leads to an accelerated movement of the trial implant part 10 in the direction toward the connecting cone 26 due to the advancing force acting on said first trial implant part 10, such that the cone base 46 is also able to strike against the cone end face 54 and cause a corresponding clicking or striking noise.

The recess 70, which weakens the sleeve wall 38 somewhat and thus makes it more elastic, improves an elastic deformation and thus also a pivoting of the second latching elements 78 back into their undeformed starting position when they engage behind the connecting cone 26. Thus, despite the first latching elements 76 sliding along the outside of the connecting cone 26, a sufficient acceleration of the first trial implant part 10 is possible for producing a clicking noise.

The described modular trial implant system 12, in particular the first trial implant part 10, allows a surgeon to easily handle the same and enables, in particular, a secure connection of the first and second trial implant parts 10 and 14, 16 or alternatively the first trial implant part 10 with an implant part to one another because, as explained, the surgeon receives acoustic feedback both when coupling the first trial implant part 10 to the shorter connecting cone 24 and to the longer connecting cone 26.

1. A modular trial implant system comprising at least one first trial implant part, which is configured to releasably connect to at least one second trial implant part and/or an implant part to form at least one first trial joint implant part, wherein the at least one first trial implant part comprises a cone receptacle for accommodating a connecting cone of the at least one second trial implant part or the at least one implant part, wherein the cone receptacle defines an implant longitudinal axis, wherein the at least one first trial implant part comprises at least two first latching elements associated with one another and at least two second latching elements associated with one another, wherein the at least two first latching elements and the at least two second latching elements are arranged or formed axially offset in the cone receptacle relative to the implant longitudinal axis for latchingly engaging behind differently long connecting cones of second trial implant parts or implant parts, wherein the at least one first trial implant part comprises a receiving sleeve defining the cone receptacle, wherein the receiving sleeve comprises a sleeve wall that surrounds the implant longitudinal axis and delimits the cone receptacle, and wherein the sleeve wall is of indentation-free and/or perforation-free configuration at least in a first region between the at least two first latching elements and at least in a second region between the at least two second latching elements.

2. The modular trial implant system according to claim 1, wherein the sleeve wall is completely of indentation-free and/or perforation-free configuration.

3. The modular trial implant system according to claim 1, wherein the cone receptacle comprises an insertion opening for inserting the connecting cone, and wherein the insertion opening is delimited by a circular ring-shaped, uninterrupted insertion opening rim.

4. The modular trial implant system according to claim 1, wherein the cone receptacle conically tapers toward a cone base and wherein the cone base extends transversely to the implant longitudinal axis.

5. The modular trial implant system according to claim 1, wherein at least one of:
a) the at least two first latching elements extend in a circumferential direction relative to the implant longitudinal axis;
b) the at least two first latching elements are arranged or formed evenly distributed over a circumference of the sleeve wall;
c) the at least two second latching elements extend in the circumferential direction relative to the implant longitudinal axis; and
d) the at least two second latching elements are arranged or formed evenly distributed over a circumference of the sleeve wall.

6. The modular trial implant system according to claim 1, wherein at least one of:
a) the at least two first latching elements define a first latching element plane and the first latching element plane extends transversely to the implant longitudinal axis;
b) the at least two second latching elements define a second latching element plane and the second latching element plane extends transversely to the implant longitudinal axis;
c) the at least two first latching elements are configured in the form of first latching projections pointing toward the implant longitudinal axis; and
d) the at least two second latching elements are configured in the form of second latching projections pointing in the direction toward the implant longitudinal axis.

7. The modular trial implant system according to claim 1, wherein the at least two first latching elements and the at least two second latching elements are arranged or formed offset from one another in a circumferential direction in such a way that, in the circumferential direction, each first latching element is arranged or formed between two second latching elements and each second latching element is arranged or formed between two first latching elements.

8. The modular trial implant system according to claim 1, wherein at least one of:
a) two first latching elements are provided and arranged or formed diametrically opposed to one another relative to the implant longitudinal axis;
b) two second latching elements are provided and arranged or formed diametrically opposed to one another relative to the implant longitudinal axis.

9. The modular trial implant system according to claim 1, wherein the at least two first latching elements extend over a first circumferential angle relative to the implant longitudinal axis, wherein the at least two second latching elements extend over a second circumferential angle relative to the implant longitudinal axis, and wherein the first circumferential angle is greater than the second circumferential angle.

10. The modular trial implant system according to claim 1, wherein the cone receptacle defines an inner wall surface, wherein a first portion that is hollow-cylindrical relative to the implant longitudinal axis is formed on the inner wall surface, and wherein the at least two first latching elements are formed on the first hollow-cylindrical portion.

11. The modular trial implant system according to claim 1, wherein a groove extends circumferentially relative to the implant longitudinal axis and is arranged or formed on the cone receptacle, and wherein the groove is arranged or formed between the at least two first latching elements and the at least two second latching elements.

12. The modular trial implant system according to claim 1, wherein the sleeve wall comprises a first sleeve portion and an adjoining second sleeve portion, wherein the cone receptacle tapers from the second sleeve portion toward the first sleeve portion, wherein the at least two first and second latching elements are arranged or formed exclusively on the second sleeve portion, and wherein a first wall thickness of the first sleeve portion is greater than a second wall thickness of the second sleeve portion.

13. The modular trial implant system according to claim 1, wherein the at least one first trial implant part is made of a plastic.

14. The modular trial implant system according to claim 1, further comprising at least one second trial implant part or at least one implant part and wherein the at least one second trial implant part or the at least one implant part comprises a connecting cone that is insertable into the cone receptacle.

15. The modular trial implant system according to claim 1, wherein at least one of:
a) the at least one first trial implant part has a spherical or ball-shaped trial joint head;
b) the modular trial implant system comprises at least one artificial trial joint with a first trial joint implant part and a second trial joint implant part that cooperates with said first trial joint implant part in a jointed manner; and
c) the at least one first trial implant part and/or the at least one second trial implant part or the implant part comprise a one-piece configuration.
